(19) United States
(12) Patent Application Publication
(10) Pub. No.: US 2022/0023177 A1
(43) Pub. Date: Jan. 27, 2022

(54) CLEANSING COMPOSITION

(71) Applicant: Shiseido Company, Ltd., Tokyo, (JP)

(72) Inventor: Tomoyuki KARIYA, Tokyo (JP)

(73) Assignee: Shiseido Company, Ltd., Tokyo, (JP)

(21) Appl. No.: 17/296,927

(22) PCT Filed: Nov. 1, 2019
(86) PCT No.: PCT/JP2019/043086

§ 371 Date: May 25, 2021

(30) Foreign Application Priority Data
Nov. 26, 2018 (JP) 2018-220595

(51) Int. Cl. A61K 8/41; A61K 8/34; A61K 8/37; A61K 8/31; A61K 8/42; A61K 8/891; A61Q 19/10

(52) U.S. Cl. CPC A61K 8/41; A61K 8/345; A61K 8/37; A61K 8/31; A61K 2800/805; A61K 8/891; A61Q 19/10; A61K 2800/591; A61K 8/42

(57) ABSTRACT

A cleansing composition is a dye cleansing composition for removing a dye from the skin. The cleansing composition contains trialkylamine oxide.

### RELATED APPLICATION

The present application is based upon and claims the benefit of priority from Japanese Patent Application No. 2018-220595 filed on Nov. 26, 2018, the entire contents of which are incorporated herein by reference.

### TECHNICAL FIELD

The present disclosure relates to a cleansing composition for removing a dye. For example, the present disclosure relates to a dye cleansing composition for removing a dye from the skin.

### BACKGROUND ART

Cosmetic components include aqueous components and oily components. When cleansing off cosmetics applied to the face, both aqueous components and oily components can be removed if cleansing is performed using separate cleansing agents for aqueous cosmetics and oily cosmetics. It is, however, troublesome to perform cleansing two or more times. To address this, for example, Patent Literature 1 discloses a cleansing cosmetic for removing both aqueous and oily soils.

The cleansing cosmetic disclosed in Patent Literature 1 contains potassium N-cocoyl glycinate and lauryl dimethylamine oxide or stearyl dimethylamine oxide.

### CITATION LIST

### Patent Literature

Patent Literature 1: Japanese Unexamined Patent Publication No. H11-199437A

### SUMMARY OF INVENTION

### Technical Problem

The following analysis can be made from the perspective of the present disclosure.

Cosmetics containing dyes are known in the art. In the present disclosure, such cosmetics are referred to as “tinted cosmetics.” Examples of tinted cosmetics may include lip cosmetics, such as lip tints or tinted lipsticks, that are suppressed from losing color. With tinted cosmetics, staining/tinting is performed by making a dye penetrate to the stratum corneum of the skin such as the lips, thereby suppressing color loss. The penetration of dye to the stratum corneum, however, makes it difficult to sufficiently remove tinted cosmetics with ordinary cleansing agents for cleansing off cosmetics applied to the skin, because it is thought that the dye chemically bonds with protein. Thus, to dilute the color of a tinted cosmetic on the outer appearance of the skin, or preferably, to completely remove the color of a tinted cosmetic from the skin, it is necessary to use a cleansing agent intended for tinted cosmetics.

Typically, acidic dyes (anionic dyes) are used for dyes in tinted cosmetics. Acidic dyes are thought to bond with proteins in the skin by ionic interaction. It is therefore conceivable to blend, for example, disodium cocoamphodiacetate as a cleaning agent component for cleansing tinted cosmetics. Unfortunately, disodium cocoamphodiacetate is highly irritant to the skin; thus, disodium cocoamphodiacetate is not preferable for cleansing where the component remains on the skin, and it needs to be rinsed off with water when removing tinted cosmetics. Stated differently, cleansers containing components that are highly irritant to the skin cannot be used as leave-on-type cleansers that are capable of cleansing off cosmetics simply by wiping, without the need to be rinsed off with water.

The cleansing cosmetic disclosed in Patent Literature 1 contains potassium N-cocoyl glycinate, which is highly irritant to the skin, and thus cannot be used as a leave-on-type cleanser.

Thus, there is a demand for a cleansing agent having excellent cleansability against tinted cosmetics and also being usable as a leave-on-type composition.

### Solution to Problem

According to a first aspect of the present disclosure, a dye cleansing composition for removing a dye from the skin is provided. The cleansing composition comprises a trialkylamine oxide represented by Chem. 1.

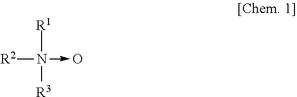

In the chemical formula represented by Chem. 1, two of R¹, R², and R³ are each a C₁₋₄ alkyl group, and one is a C₁₂₋₂₁ alkyl group.

### Advantageous Effects of Invention

The cleansing composition of the present disclosure has excellent cleansability against tinted cosmetics.

The cleansing composition of the present disclosure can be used as a leave-on-type composition.

### DESCRIPTION OF EMBODIMENTS

Preferred modes according to the aforementioned aspects will be described below.

According to a preferred mode of the above first aspect, the dye is an acidic dye.

According to a preferred mode of the above first aspect, a content by percentage of the trialkylamine oxide is from 0.2 to 4% by mass with respect to the mass of the composition.

According to a preferred mode of the above first aspect, the composition comprises an oil phase and an aqueous phase. In a stationary state, the oil phase and the aqueous phase are primarily not emulsified.

According to a preferred mode of the above first aspect, a content by percentage of the oil phase is from 20 to 80% by mass with respect to the mass of the composition. A content by percentage of the aqueous phase is from 20 to 80% by mass with respect to the mass of the composition.

According to a preferred mode of the above first aspect, the aqueous phase has a pH of from 4 to 8.

According to a preferred mode of the above first aspect, a content by percentage of a surfactant is 0.2% by mass or less with respect to the mass of the composition.

According to a preferred mode of the above first aspect, the cleansing composition further comprises an alkylbetaine-type surfactant.

According to a preferred mode of the above first aspect, the cleansing composition further comprises from 1 to 20% by mass of a water-soluble alcohol with respect to the mass of the composition.

According to a preferred mode of the above first aspect, the cleansing composition further comprises from 15 to 50% by mass of a volatile oily component with respect to the mass of the composition.

According to a preferred mode of the above first aspect, the volatile oily component is at least one selected from the group consisting of hydrocarbon oils, ester oils, and silicone oils.

According to a preferred mode of the above first aspect, the cleansing composition further comprises from 0.1 to 5% by mass of a salt with respect to the mass of the composition.

According to a preferred mode of the above first aspect, the cleansing composition further comprises from 0.05 to 5% by mass of a polyol derivative with respect to the mass of the composition. The polyol derivative is a glycerin derivative represented by Chem. 2 and/or a glycol derivative represented by Chem. 3.

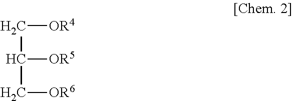

In the chemical formula represented by Chem. 2, one of R⁴, R⁵, and R⁶ is a C4-15 alkyl group, alkenyl group or acyl group, and two are each a hydrogen atom.

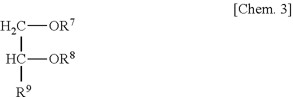

In the chemical formula represented by Chem. 3, one of R⁷ and R⁸ is a C₁₀₋₂₀ alkyl group, alkenyl group or acyl group, and the other is a hydrogen atom. R⁹ is a C₁₋₄ alkyl group, alkenyl group or acyl group, or a hydrogen atom.

According to a preferred mode of the above first aspect, the glycerin derivative includes ethylhexylglycerin and/or hexylglycerin.

According to a preferred mode of the above first aspect, the glycol derivative includes at least one selected from the group consisting of propylene glycol laurate, propylene glycol stearate, and propylene glycol isostearate.

According to a preferred mode of the above first aspect, the cleansing composition is a cleansing composition for cosmetics.

According to a preferred mode of the above first aspect, the cleansing composition is a leave-on-type composition.

In the following description, POE is an abbreviation of polyoxyethylene, and POP is an abbreviation of polyoxypropylene. The number in parentheses after POE or POP indicates the average number of moles of POE groups or POP groups added in the compound in question.

In the present disclosure, “substantial amount” refers to an amount capable of bringing about effects due to addition of the compound in question.

The dye cleansing composition according to a first embodiment of the present disclosure will be described below. The dye cleansing composition according to the first embodiment can be suitably used for removing, from the skin, dyes staining the skin. For example, the dye cleansing composition according to the first embodiment can be used for removing acidic dyes that bond with proteins in the skin by chemical interaction (e.g., ionic interaction).

(A) Trialkylamine Oxide:

A cleansing composition according to a first embodiment contains (A) a trialkylamine oxide. The trialkylamine oxide may be water-soluble or water-insoluble (oil-soluble). The trialkylamine oxide may be a mixture of a water-soluble trialkylamine oxide and an oil-soluble trialkylamine oxide. In cases where the composition is an aqueous composition, it is preferred that the trialkylamine oxide is water-soluble. Solubility to water or oily components can be adjusted by adjusting the length of the alkyl group(s) in the trialkylamine oxide.

The trialkylamine oxide may have a structure represented by Chem. 4. R¹, R², and R³ may each be a linear alkyl group or a branched-chain alkyl group.

Any two of R¹, R², and R³ (e.g., R¹ and R²) may each be a C₁₋₄ alkyl group. The remaining one of R¹, R², and R³ (e.g., R³) may be a C₁₂₋₂₁ alkyl group (e.g., a C₂₄ alkyl group). The trialkylamine oxide may be, for example, lauryl dimethylamine oxide and/or stearyl dimethylamine oxide, wherein R¹ and R² are each a methyl group, and R³ is a dodecyl group (lauryl group) and/or an octadecyl group (stearyl group).

Water-soluble components (A) can be blended, for example, to water-based compositions and facial cleansing compositions. For example, stearyl dimethylamine oxide, which is a solid at atmospheric temperature, can be blended to emulsion-type compositions. For example, lauryl diethylamine oxide, which has higher water solubility, can be blended to water-based compositions.

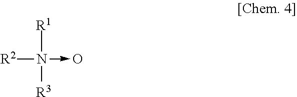

In the first embodiment, the content by percentage of component (A) with respect to the mass of the composition is preferably 0.2% by mass or greater, more preferably 0.4% by mass or greater, further preferably 0.6% by mass or greater, further preferably 0.8% by mass or greater, further preferably 1% by mass or greater, further preferably 1.2% by mass or greater, further preferably 1.5% by mass or greater. If the content of component (A) is less than 0.2% by mass, cleansability against tinted components may deteriorate. The content by percentage of component (A) with respect to the mass of the composition may be, for example, 5% by mass or less, 4% by mass or less, 3% by mass or less, or 2% by mass or less.

According to the first embodiment, dyes (e.g., tinted cosmetics) staining the skin can be cleansed off from the skin.

The dye cleansing composition according to the first embodiment can also be used as a leave-on-type cleansing agent that does not need to be washed off.

In the cleansing composition according to the first embodiment, component (A) may be dissolved in either the oil phase or the aqueous phase. For example, in cases where component (A) is oil-soluble, component (A) can be added to the aqueous phase by dissolving component (A) in a water-soluble alcohol.

The cleansing composition according to the first embodiment may further contain at least one selected from the group consisting of (B) oily components, (C) surfactants, (D) water, (E) water-soluble alcohols, and (F) polyol derivatives.

(B) Oily Component:

In cases where component (A) is oil-soluble, it is preferred that the oily component is capable of dissolving component (A). Further, it is preferred that component (B) has good cleansability against oily cosmetics such as mascaras. It is preferred that component (B) is primarily in a liquid state at room temperature.

Examples of oily components may include liquid oils, solid fats, waxes, hydrocarbons, higher fatty acids, higher alcohol, synthetic ester oils, and silicone oils.

Examples of the liquid oil that may be used may include avocado oil, camellia oil, turtle oil, macadamia nut oil, corn oil, mink oil, olive oil, rapeseed oil, egg yolk oil, sesame oil, par chic oil, wheat germ oil, southern piece oil, castor oil, linseed oil, safflower oil, cotton seed oil, perilla oil, soybean oil, groundnut oil, brown real oil, torreya oil, rice bran oil, Chinese tung oil, Japanese tung oil, jojoba oil, germ oil, triglycerol, and the like.

Examples of the solid fat that may be used may include cacao butter, coconut oil, horse fat, hydrogenated coconut oil, palm oil, beef tallow, sheep tallow, hydrogenated beef tallow, palm kernel oil, lard, beef bones fat, Japan wax kernel oil, hardened oil, hoof oil, Japan wax, hydrogenated caster oil, and the like.

Examples of the waxes that may be used may include beeswax, candelilla wax, cotton wax, carnauba wax, bayberry wax, insect wax, spermaceti, montan wax, bran wax, lanolin, kapok wax, lanolin acetate, liquid lanolin, sugarcane wax, lanolin fatty acid isopropyl ester, hexyl laurate, reduced lanolin, jojoba wax, hardened lanolin, shellac wax, POE lanolin alcohol ether, POE lanolin alcohol acetate, POE cholesterol ether, lanolin fatty acid polyethylene glycol, POE hydrogenated lanolin alcohol ether, and the like.

Examples of the hydrocarbon oils that may be used may include liquid paraffin, ozocerite, squalane, pristane, paraffin, ceresin. squalene, vaseline, microcrystalline wax, n-hexane, isohexane, cyclehexane, n-octane, isooctane, n-nonane, n-decane, isododecane, isohexadecane, and the like.

Examples of the higher fatty asid that may be used may include lauric acid, myristic acid, palmitic acid, stearic acid, behenic acid, oleic acid, undecylenic acid, tallic acid, isostearic acid, linoleic acid, linolenic acid, eicosapentaenoic acid (EPA), docosahexaenoic acid (DHA) and the like.

Examples of the higher alcohol that may be used may include linear alcohol (such as lauryl alcohol, cetyl alcohol, stearyl alcohol, behenyl alcohol, myristyl alcohol, oleyl alcohol, and cetostearyl alcohol); branched-chain alcohol (such as monostearylglycerin ether (batyl alcohol), 2-decyltetradecinol, lanolin alcohol, cholesterol, phytosterol, hexyldodecanol, isostearyl alcohol, and octyldodecanol) and the like.

Examples of the synthesis ester oils that may be used may include isopropyl myristate, cetyl octanoate, octyldodecyl myristate, isopropyl palmitate, butyl stearate, hexyl laurate, myristyl myristate, decyl oleate, hexyldecyl dimethyl octanoate, cetyl lactate, myristyl lactate, lanolin acetate, isocetyl stearate, isocetyl isostearate, cholesteryl 12-hydroxy stearate, ethylene glycol di-2-ethyl hexanoate, di-penta erythritol fatty acid ester, N-alkyl glycol monoisostearate, neopentyl glycol dicaprate, diisostearyl malate, glyceryl di-2-heptyl undecanoate, trimethyrol propane tri-2-ethyl hexanoate, trimethyrol propane triisostearate, pentaerythritol tetra-2-ethyl hexanoate, glyceryl tri-2-ethyl hexanoate, glyceryl trioctanoate, glyceryl triisopalmitate, trimethyrol propane triisostearate, cetyl 2-ethylhexanoate, 2-ethylhexyl palmitate, glyceryl trimyristate, glyceride tri-2-heptyl undecanoate, castor oil fatty acid methyl ester, oleyl oleate, acetoglyceride, 2-heptylundecyl palmitate, diisobutyl adipate, N-lauroyl-L-glutamic acid-2-octyldodecyl ester, di-2-heptylundecyl adipate, ethyl laurate, di-2-ethylhexyl sebacate, 2-hexyldecyl myristate, 2-hexyldecyl palmitate, 2-hexyldecyl adipate, diisopropyl sebacate, 2-ethylhexyl succinate, triethyl citrate, and the like.

Examples of the silicone oil may include silicone compounds such as dimethylpolysiloxane, methylhydrogenpolysiloxane, methylphenylpolysiloxane, stearoxymethylpolysiloxane, polyether-modified organopolysiloxane, fluoroalkyl/polyoxyalkylene co-modified organopolysiloxane, alkyl-modified organopolysiloxane, terminal-modified organopolysiloxane, fluorine-modified organopolysiloxane, amino-modified organopolysiloxane, silicone gel, acrylic silicone, trimethylsiloxysilicic acid, silicone RTV rubber and the like.

Among the aforementioned oily components, isododecane, dimethyl polysiloxane, cetyl octanoate, and the like are preferred because of excellent cleansability against oily cosmetics.

The content by percentage of component (B) with respect to the mass of the composition is preferably 20% by mass or greater, more preferably 25% by mass or greater, further preferably 28% by mass or greater. If the content of component (B) is less than 20% by mass, component (A) may precipitate out, which may deteriorate the transparency of liquid compositions. The content by percentage of component (B) with respect to the mass of the composition may be, for example, 80% by mass or less, 70% by mass or less, or 60% by mass or less.

Among the component (B), the content of volatile oily component(s) with respect to the mass of the composition is preferably 15% by mass or greater, more preferably 20% by mass or greater, further preferably 25% by mass or greater. If the content of volatile oily component(s) is less than 15% by mass, cleansability against oily cosmetics may deteriorate. The content by percentage of volatile oily component(s) with respect to the mass of the composition may be, for example, 50% by mass or less, 45% by mass or less, or 40% by mass or less.

(C) Surfactant:

Component (C) may be added, for example, to temporarily emulsify the cleansing composition at the time of use in an oil-water two-layer-type cleansing composition. Examples of component (C) may include the following surfactants.

(C1) Anionic Surfactant:

Examples of the anionic surfactants that may be used may include fatty acid soap (such as sodium laurate, and sodium palmitate); higher alkyl sulfate ester salt (such as sodium lauryl sulfate, and potassium lauryl sulfate); alkyl ether sulfate ester salt (such as POE-lauryl sulfate triethanolamine, and sodium POE-lauryl sulfate); N-acyl sarcosinic acid (such as sodium lauroyl sarcocinate); higher fatty acid amide sulfonate (such as sodium N-stearoyl-N-methyltaurate, sodium N-myristoyl-N-methyltaurate, sodium methyl cocoyl taurate, and sodium laurylmethyl taurate); phosphate ester salt (sodium POE-oleylether phosphate, POE-stearylether phosphate, potassium cetyl phosphate); sulfosuccinate (such as sodium di-2-ethylhexyl sulfosuccinate, sodium monolauroyl monoethanolamide polyethylene sulfosuccinate, and sodium lauryl polypropylene glycol sulfosuccinate); alkylbenzene sulfonate (such as sodium linear dodecylbenzene sulfonate, triethanolamine linear dodeylbenzene sulfonate, and linear dodecylbenzene sulfonate); higher fatty acid ester sulfate ester salt (such as sodium hydrogenated gryceryl cocoate sulfate); N-acyl glutamate (such as monosodium N-lauroyl glutamate, disodium N-stearoyl glutamate, and monosodium N-myristoyl-L-glutamate); sulfonated oil (such as Turkey red oil); POE-alkyl ether carboxylic acid; POE-alkyl aryl ether carboxylate; α-olefine sulfonate; higher fatty acid ester sulfonate; secondary alcohol sulfate ester salt; higher fatty acid alkylolamide sulfate ester salt; sodium lauroyl monoethanolamide succinate; N-palmitoyl asparaginate ditriethanolamine; sodium casein; and the like.

(C2) Cationic Surfactant:

Examples of the cationic surfactants may include alkyltrimethyl ammonium salt (such as stearyltrimethyl ammonium chloride, lauryltrimethyl ammonium chloride); alkylpyridinium salt (such as cetylpyridinium chloride); dialkyldimethyl ammonium salt (such as distearyldimethyl ammonium chloride); poly (N,N′-dimethyl-3,5-methylenepiperidinium) chloride; alkyl quaternary ammonium salt; alkyldimethylbenzyl ammonium salt; alkylisoquinolinium salt; dialkylmorphonium salt; POE alkylamine; alkylamine salt; polyamine fatty acid derivative; amyl alcohol fatty acid derivative; benzalkonium chloride; benzethonium chloride, amino acid-based cationic surfactant (such as ethyl L-cocoyl arginine DL-pyrrolidonecarboxylic acid salt) and the like.

(C3) Amphoteric Surfactant:

Examples of the amphoteric surfactant that may be used may include: imidazoline-based amphoteric surfactant (such as sodium 2-undecyl-N,N,N-(hydroxyethylcarboxymethyl)-2-imidazoline and 2-cocoyl-2-imidazolinium hydroxide-1-carboxyethyloxy disodium salt); and betaine-based surfactant (such as 2-heptadecyl-N-carboxymethyl-N-hydroxyethyl imidazolinium betaine, lauryl dimethylaminoacetic acid betaine, alkyl betaine, amidobetaine, and sulfobetaine).

(C4) Hydrophilic Nonionic Surfactant:

Examples of the hydrophilic nonionic surfactants that may be used may include POE sorbitan fatty acid ester (such as POE sorbitan monooleate, POE sorbitan monostearate, POE sorbitan monooleate, POE sorbitan tetraoleate); POE sorbit fatty acid ester (such as POE sorbit monolaurate, POE sorbit monooleate, POE sorbit pentaoleate, POE sorbit monostearate), POE glyceryl fatty acid ester (such as POE monooleate such as POE glyceryl monostearate, POE glyceryl monoisostearate, POE glyceryl triisostearate); POE fatty acid ester (such as POE distearate, POE monodioleate, ethyleneglycol distearate); POE alkyl ether (such as POE lauryl ether, POE oleyl ether, POE stearyl ether, POE behenyl ether, POE-2-octyldodecyl ether, POE cholestanol ether); puluronic type (such as Puluronic), POE/POP alkyl ethers (such as POE/POP cetyl ether, POE/POP 2-decyltetradecyl ether, POE/POP monobutyl ether, POE/POP hydrogenated lanoline, POE/POP glycerin ether); tetra POE/tetra POP ethylenediamine condensation products (such as Tetronic); POE castor oil hydrogenated castor oil derivative (such as POE caster oil, POE hydrogenated caster oil, POE hydrogenated caster oil monoisostearate, POE hydrogenated castor oil triisostearate, POE hydrogenated caster oil monopyroglutamate monoisostearate diester, POE hydrogenated oil maleate); POE beeswax/lanoline derivative (such as POE sorbitol beeswax); alkanolamide (such as coconut oil fatty acid diethanolamide, lauric acid monoethanolamide, fatty acid isopropanolamide); POE propyleneglycol fatty acid ester; POE alkyl amines; POE fatty acid amide; sucrose fatty acid ester; alkylethoxydimethylamine oxide; trioleyl phosphoric acid and the like.

(C5) Lipopholic Nonionic Surfactant:

Examples of the lipophilic nonionic surfactants may include sorbitan fatty acid ester (such as sorbitan monooleate, sorbitan monoisostearate, sorbitan monolaurate, sorbitan monopalmitate, sorbitan monostearate, sorbitan sesquioleate, sorbitan trioleate, diglycerol sorbitan penta-2 ethylhexylate, diglycerol sorbitan tetra-2 ethylhexylate, etc); glyceryl polyglyceryl fatty acid (such as glyceryl monocotton oil fatty acid, glyceryl monoerucate, glyceryl sesquioleate, glyceryl monostearate, glyceryl α, α′-oleate pyroglutamate, glyceryl monostearate malate, etc); propylene glycol fatty acid ester (such as propylene glycol monostearate, etc); hydrogenated caster oil derivative; glyceryl alkyl ether, and the like.

In cases where the cleansing composition is to be made usable as a leave-on-type composition, it is even preferred that the surfactant is, for example, alkyl betaine, ethyl L-cocoyl arginine DL-pyrrolidonecarboxylic acid salt (PCA ethyl cocoyl arginate), benzalkonium chloride, or the like, from the viewpoint of causing less irritation to the skin.

In cases where the cleansing composition is to be made usable as a leave-on-type composition, the content by percentage of component (C) may be 0.02% by mass or greater, 0.05% by mass or greater, or 0.07% by mass or greater, with respect to the mass of the composition. If the content of component (C) is less than 0.02% by mass, the cleansability against tint components may deteriorate. The content by percentage of component (C) with respect to the mass of the composition may be, for example, 0.3% by mass or less, 0.2% by mass or less, 0.15% by mass or less, or 0.1% by mass or less.

In cases where the cleansing composition is not to be used as a leave-on-type composition, the content by percentage of component (C) may be 1% by mass or greater, 1.5% by mass or greater, or 2% by mass or greater, with respect to the mass of the composition. The content by percentage of component (C) with respect to the mass of the composition may be, for example, 5% by mass or less, 3% by mass or less, or 2% by mass or less.

(D) Water:

Examples of water usable herein may include water used for cosmetics, quasi-pharmaceutical products, or the like, and usable examples may include purified water, ion-exchanged water, or tap water.

The content by percentage of component (D) with respect to the mass of the composition is preferably 20% by mass or greater, more preferably 30% by mass or greater, further preferably 40% by mass or greater. If the content of component (D) is less than 20% by mass, the emulsification duration after shaking may be insufficient in cases where the composition is an oil-water two-layer separation-type composition. The content by percentage of component (D) with respect to the mass of the composition may be, for example, 80% by mass or less, 70% by mass or less, or 60% by mass or less.

(E) Water-Soluble Alcohol:

Examples of the water-soluble alcohol may include at least one selected from, for example, lower alcohols, polyols, polyol polymers, divalent alcohol alkyl ethers, divalent alcohol alkyl ethers, divalent alcohol ether esters, glycerin monoalkyl ethers, sugar alcohols, monosaccharides, oligosaccharides, polysaccharides, and derivatives thereof.

Examples of the lower alcohol may include ethanol, propanol, isopropanol, isobutyl alcohol, t-butyl alcohol, and the like.

Examples of the polyhydric alcohol may include dihydric alcohol (such as ethylene glycol, propylen glycol, trimethylene glycol, 1,2-butylene glycol, 1,3-butylene glycol, tetramethylene glycol, 2,3-butylene glycol, pentamethylene glycol, 2-butene-1,4-diol, hexylene glycol, octylene glycol, etc); trihydric alcohol (such as glycerin, trimethylolpropane, etc); tetrahydric alcohol (such as such as pentaerythritol such as 1,2,6-hexanetriol, etc); pentahydric alcohol (such as xylitol, etc); hexahydric alcohol (such as sorbitol, mannitol, etc); polyhydric alcohol polymer (such as diethylene glycol, dipropylene glycol, triethylene glycol, polypropylene glycol, tetraethylene glycol, diglycerin, polyethylene glycol, triglycerin, tetraglycerin, polyglycerin, etc); dihydric alcohol alkyl ethers (such as ethylene glycol monomethyl ether, ethylene glycol monoethyl ether, ethylene glycol monobutyl ether, ethylene glycol monomphenyl ether, ethylene glycol monohexyl ether, ethylene glycol mono2-methylhexyl ether, ethylene glycol isoamyl ether, ethylene glycol benzil ether, ethylene glycol isopropyl ether, ethylene glycol dimethyl ether, ethylene glycol diethyl ether, ethylene glycol dibutyl ether, etc); dihydric alcohol alkyl ethers (such as diethylene glycol monomethyl ether, diethylene glycol monoethyl ether, diethylene glycol monobutyl ether, diethylene glycol dimethyl ether, diethylene glycol diethyl ether, diethylene glycol butyl ether, diethylene glycol methylethyl ether, triethylene glycol monomethyl ether, triethylene glycol monoethyl ether, propylene glycol monomethyl ether, propylene glycol monoethyl ether, propylene glycol monobutyl ether, propylene glycol isopropyl ether, dipropylene glycol methyl ether, dipropylene glycol ethyl ether, dipropylene glycol butyl ether, etc); dihydric alcohol ether ethers (such as ethylene glycol monomethyl ether acetate, ethylene glycol monoethyl ether acetate, ethylene glycol monobutyl ether acetate, ethylene glycol monophenyl ether acetate, ethylene glycol diadipate, ethylene glycol disaccinate, diethylene glycol monoethyl ether acetate, diethylene glycol monobutyl ether acetate, propylene glycol monomethyl ether acetate, propylene glycol monoethyl ether acetate, propylene glycol monopropyl ether acetate, propylene glycol monophenyl ether acetate, etc); glycerin monoalkyl ether (such as chimyl alcohol, selachyl alcohol, batyl alcohol, etc); sugar alcohol (such as sorbitol, maltitol, maltotriose, mannitol, sucrose, erythritol, glucose, fructose, starch sugar, maltose, xylitol, starch sugar hydrogenated alcohol, etc); glycolide, tetrahydrofurfuryl alcohol; POE-tetrahydrofurfuryl alcohol; POP/POE-butyl ether; tripolyoxypropylene glycerin ether; POP-glycerin ether; POP-glycerin ether phosphoric acid; POP/POE-pentaerythritol ether; polyglycerin, and the like.

Examples of the monosaccharides may include at least one selected from triose (such as D-glyceryl aldehyde, dihydroxyacetone, etc); tetrose (such as D-erythrose, D-erythrulose, D-threose, erythritol, etc); pentaose (such as L-arabinose, D-xylose, L-lyxose, D-arabinose, D-ribose, D-ribulose, D-xylulose, L-xylulose, etc); hexalose (such as D-glucose, D-talose, D-psicose, D-galactose, D-fructose, L-galactose, L-mannose, D-tagatose, etc); heptose (such as aldoheptose, heptulose, etc); octose (such as octulose, etc); deoxy sugar (such as 2-deoxy-D-ribose, 6-deoxy-L-galactose, 6-deoxy-L-mannose, etc); amino sugar (such as D-glucosamine, D-galactosamine, sialic acid, amino uronic acid, muramic acid, etc); uronic acid (such as D-glucuronic acid, D-mannuronic acid, L-guluronic acid, D-galacturonic acid, L-iduronic acid, etc) and the like.

Examples of the oligosaccharide may include at least one selected from sucrose, guntianose, umbelliferose, lactose, planteose, isolignoses, α,α-trehalose, raffinose, lignoses, umbilicin, stachyose, verbascoses, and the like.

Examples of the polysaccharide may include at least one selected from cellulose, quince seed, chondroitinsulfate, starch, galactan, dermatan sulfate, glycogen, acasia gum, heparansulfate, hyaluronan, gum tragacanth, keratan sulfate, chondoroitin, xanthan gum, mucoitin sulfate, guar gum, dextran, keratosulfate, locust bean gum, succinoglycan, caronic acid, and the like.

Examples of other polyols may include at least one polyol selected from polyoxyethylene methyl glucoside (Glucam E-10), polyoxypropylene methyl glucoside (Glucam P-10), and the like.

Among the aforementioned water-soluble alcohols, it is preferred to employ ethanol, butylene glycol, dipropylene glycol, or the like, from the viewpoint of emulsification adjustability and preservative properties.

The content by percentage of component (E) with respect to the mass of the composition is preferably 2% by mass or greater, more preferably 5% by mass or greater, further preferably 10% by mass or greater. If the content of component (E) is less than 2% by mass, preservative efficacy may be insufficient. The content by percentage of component (E) with respect to the mass of the composition may be, for example, preferably 30% by mass or less, more preferably 20% by mass or less, further preferably 15% by mass or less. If the content of component (E) exceeds 30% by mass, emulsification after shaking may be insufficient in cases where the composition is an oil-water two-layer separation-type composition.

(F) Polyol Derivative:

The cleansing composition according to the first embodiment may further contain a polyol derivative. The polyol derivative may be a glycerin derivative and/or a glycol derivative.

For the glycerin derivative, it is possible to use, for example, an alkylglyceryl ether and/or a glycerin ester, and particularly a monoalkylglyceryl ether. For the glycerin derivative, it is possible to use one of compounds represented by Chem. 5 below. In the chemical formula represented by Chem. 5, one of R⁴, R⁵, and R⁶ may be an alkyl group, an alkenyl group, or an acyl group, and the other two may each be a hydrogen atom. It is preferred that the alkyl group, alkenyl group or acyl group contains 4 or more carbon atoms. If the number of carbon atoms is fewer than 4, a sufficient cleansing action may not be obtained. The number of carbon atoms in the alkyl group, alkenyl group or acyl group may preferably be 15 or fewer, more preferably 12 or fewer. If the number of carbon atoms exceeds 15, a sufficient cleansing action may not be obtained. The alkyl group, alkenyl group or acyl group may be linear or may be branched.

Examples of the glycerin derivative may include ethylhexylglycerin (octoxyglycerin), hexylglycerin, glyceryl isooctanoate, polyglyceryl-2 laurate, glyceryl monooctanoate, and the like. Among the above, from the viewpoint of eliminating liquid droplets adhering to the inner surface of a container, it is preferred to use ethylhexylglycerin having a 2-ethylhexyl group and/or hexylglycerin having a hexyl group. An example of a commercially available product of ethylhexylglycerin may include Sensiva SC50 (from Schulke & Mayr GmbH).

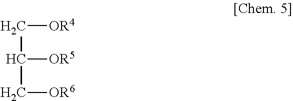

For the glycol derivative, it is possible to use, for example, a glycol ester and/or a glycol ether. For the glycol derivative, it is possible to use, for example, a propylene glycol fatty acid ester and/or a propylene glycol ether, and particularly a propylene glycol mono-fatty acid ester. For the glycol derivative, it is possible to use one of compounds represented by Chem. 6. In the chemical formula represented by Chem. 6, one of R⁷ and R⁸ may be a C₁₀₋₂₀ alkyl group, alkenyl group or acyl group, and the other may be a hydrogen atom. R⁹ may be a C₁₋₄ alkyl group, alkenyl group or acyl group, or a hydrogen atom. The alkyl group, alkenyl group or acyl group may be linear or may be branched.

Examples of the glycol derivative may include propylene glycol laurate, propylene glycol stearate, propylene glycol isostearate, and the like.

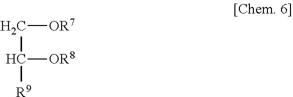

The glycerin derivative and the glycol derivative may be present in either the oil phase or the aqueous phase.

Component (F) is capable of suppressing liquid droplets of the cleansing composition from remaining on the inner surface of a container. For example, particularly in cases where the cleansing composition is an oil-water two-layer-type liquid, liquid droplets adhering to the inner surface of a transparent container in a space above the liquid can be inhibited from being seen through the container, thereby improving product appearance.

In cases where the cleansing composition is an oil-water two-layer-type composition, component (F) also acts to clearly define the interface between the oil phase and the aqueous phase at the time of re-separation of the oil phase and the aqueous phase that occurs by leaving the composition to stand in a stationary state after temporary emulsification. In this way, the appearance of the oil-water separation-type composition as seen through a container can be further improved.

In cases where the cleansing composition has an oil-water two-layer structure and an oil-soluble component (A) is blended to the aqueous phase, component (F) can improve the stability of the cleansing composition after temporary emulsification. Further, the glycerin derivative can improve the cleansability of the cleansing composition.

The content by percentage of component (F) with respect to the mass of the composition is preferably 0.06% by mass or greater, more preferably 0.07% by mass or greater, further preferably 0.08% by mass or greater. The content by percentage of the polyol derivative with respect to the mass of the composition may be 0.1% by mass or greater, 0.2% by mass or greater, or 0.5% by mass or greater. If the content of the polyol derivative is less than 0.06% by mass, the aforementioned actions may not be obtained sufficiently. The content by percentage of component (F) with respect to the mass of the composition is preferably 1.8% by mass or less, more preferably 1.5% by mass or less, further preferably 1.2% by mass or less. The content by percentage of the polyol derivative with respect to the mass of the composition may be 1% by mass or less, 0.8% by mass or less, or 0.5% by mass or less. If the content of the polyol derivative exceeds 1.8% by mass, the interface between the oil phase and the aqueous phase may become unclear.

(G) Salt:

The cleansing composition according to the first embodiment may further contain a salt in cases where the cleansing composition contains water or an aqueous phase. Particularly in cases where the cleansing composition is an oil-water two-layer-type composition, it is preferred that the cleansing composition contains a salt. Adding a salt makes it possible to adjust the re-separation speed after temporary emulsification.

Component (G) may be an inorganic salt or an organic salt, so long as it is hypoirritant to the skin. Examples of component (G) may include sodium chloride, potassium chloride, sodium citrate, sodium edetate, and the like.

The content by percentage of component (G) with respect to the mass of the composition is preferably 0.1% by mass or greater, more preferably 0.2% by mass or greater, further preferably 0.3% by mass or greater. If the content of component (G) is less than 0.1% by mass, the aforementioned actions may not be obtained sufficiently. The content by percentage of component (G) with respect to the mass of the composition is preferably 2% by mass or less, more preferably 1.5% by mass or less, further preferably 1% by mass or less. If the content of component (G) exceeds 2% by mass, emulsification after shaking may be insufficient in cases where the composition is an oil-water two-layer separation-type composition.

(H) Others:

If necessary, the cleansing composition of the present disclosure may contain other components as appropriate, such as powder bodies, moisturizers, water-soluble polymers, thickeners, film-forming agents, UV absorbers, metal ion sequestering agents, amino acids, organic amines, polymer emulsions, pH adjusters, skin nutrients, vitamins, antioxidants, antioxidant aids and perfumes, in amounts that do not inhibit the effects of the present disclosure.

The terms “powder” and “powdered component” as used herein are synonymous. The powder is not particularly limited so long as it is generally usable for cosmetic purposes, for example. Examples of the powder bodies may include inorganic powder (such as talc, kaolin, mica, sericite, muscovite, phlogopite, synthetic mica, lepidolite, biotite, vermiculite, magnesium carbonate, calcium carbonate, aluminum silicate, barium silicate, calcium silicate, magnesium silicate, strontium silicate, tungstate, magnesium, silica, zeolite, glass, barium sulfate, calcined calcium sulfate (calcined gypsum), calcium phosphate, fluorine apatite, hydroxyapatite, ceramic powder, metallic soap (such as zinc myristate, calcium palimitate, and aluminum stearate), and boron nitride, etc); organic powder (such as polyamide resin powder (nylon powder), polyethylene powder, polymethylmethacrylate powder, polystyrene powder, styrene-acrylic acid copolymer powder, benzoguanamine resin powder, poly(tetrafluroethylene) powder, and cellulose powder, silicone resin powder, silk powder, wool powder, urethane powder, etc); inorganic white family pigment (such as titanium dioxide, zinc oxide, etc); inorganic red family pigment (such as iron oxide (colcothar), iron titanate, etc); inorganic brown family pigment (such as γ-iron oxide, etc); inorganic yellow family pigment (such as yellow iron oxide, loess, etc); inorganic black family pigment (such as black iron oxide, carbon black, lower titanium oxide, etc); inorganic purple family pigment (such as manganese violet, cobalt violet, etc); inorganic green family pigment (such as chrome oxide, chrome hydroxide, cobalt titanate, etc); inorganic blue family pigment (such as ultramarine, iron blue, etc); pearl pigment (such as titanium oxide coated mica, titanium oxide coated bismuth oxychloride, titanium oxide coated talc, colored titanium oxide coated mica, bismuth oxychloride, argentine, etc); metal powder pigment (such as aluminum powder, copper powder, etc); organic pigment such as zirconium, barium, or aluminum lake (such as organic pigment such as Red No. 201, Red No. 202, Red No. 204, Red No. 205, Red No. 220, Red No. 226, Red No. 228, Red No. 405, Red No. 201, Orange No. 203, Orange No. 204, Yellow No. 205, Yellow No. 401, Blue No. 401, Red No. 3, Red No. 104, Red No. 106, Red No. 227, Red No. 230, Red No. 401, Red No. 505, Orange No. 205, Yellow No. 4, Yellow No. 5, Yellow No. 202, Yellow No. 203, Green No. 3, and Blue No. 1, etc); natural pigment (such as chlorophyll, β-carotene, etc) and the like.

Examples of the moisturizers may include polyethylene glycol, propylene glycol, glycerin, 1,3-butylene glycol, xylitol, sorbitol, maltitol, chondroitin sulfate, hyaluronic acid, mucoitin sulfate, charonic acid, atelocollagen, cholesteryl 12-hydroxystearate, sodium lactate, bile salt, dl-pyrrolidone carboxylate, alkyleneoxide derivative, short-chain soluble collagen, diglycerin (EO)PO adduct, chestnut rose extract, yarrow extract, melilot extract, and the like.

Examples of the natural water-soluble polymer may include plant-based polymer (such as gum Arabic, gum tragacanth, galactan, guar gum, locust bean gum, gum karaya, carrageenan, pectine, agar, quince seed (cydonia oblonga), algae colloid (brown algae extract), starch (rice, corn, potato, wheat), glycyrrhizic acid); microorganism based polymer (such as xanthan gum, dextran, succinoglycan, pullulan, etc), animal-based polymer (such as collagen, casein, albumin, gelatine, etc) and the like.

Examples of the semisynthetic water-soluble polymer may include starch-based polymer (such as carboxymethyl starch, methylhydroxypropyl starch, etc); cellulose-based polymer (such as methylcellulose, ethylcellulose, methylhydroxypropylcellulose, hydroxyethylcellulose, cellulose sodium sulfate, hydroxypropylcellulose, carboxymethylcellulose, sodium carboxymethyl cellulose, crystalline cellulose, cellulose powder, etc); align acid-based polymer (such as sodium alginate, propylene glycol alginate ester, etc), and the like.

Examples of the synthetic water-soluble polymer may include vinyl based polymer (such as polyvinyl alcohol, polyvinyl methyl ether, polyvinylpyrrolidone, carboxyvinylpolymer, etc); polyoxyethylene based polymer (such as polyoxyethylenepolyoxypropylene copolymer such as polyethylene glycol 20,000, 40,000 and 60,000, etc); acrylic polymer (such as sodium polyacrylate, polyethylacrylate, polyacrylamide, etc); polyethyleneimine; cationic polymer; and the like.

Examples of other thickeners may include gum arabic, carrageenan, karaya gum, tragacanth gum, carob gum, quince seed (marmelo), casein, dextrin, gelatin, sodium pectate, sodium alginate, methyl cellulose, ethyl cellulose, carboxymethyl cellulose (CMC), hydroxyethyl cellulose, hydroxypropyl cellulose, polyvinyl alcohol (PVA), polyvinylmethyl ether (PVM), PVP (polyvinyl pyrrolidone), polysodium acrylate, carboxyvinyl polymer, locust bean gum, guar gum, tamarind gum, dialkyldimethylammonium sulfate cellulose, xanthan gum, aluminum magnesium silicate, bentonite, hectorite, aluminum magnesium silicate (Veegum), sodium magnesium silicate (Laponite), silicic acid anhydride, taurate-based synthetic polymers, and acrylate-based synthetic polymers.

Examples of the film-forming agent may include an anionic film-forming agent (such as (meta)acrylic acid/(meta)acrylic acid ester copolymer, methyl vinyl ether/maleic anhydride copolymer, etc), a cationic film-forming agent (such as cationic cellulose, diallyldimethylammonium chloride polymer, diallyldimethylammonium chloride/acrylic amide copolymer, etc), a nonionic film-forming agent (such as polyvinyl alcohol, polyvinylpyrrolidone, polyvinyl acetate, polyacrylic ester copolymer, (meta)acrylamide, polymeric silicone, silicone resin, trimethylsiloxysilicate, etc), and the like.

Examples of the ultraviolet light absorbers may include benzoic acid family ultraviolet light absorber (such as p-aminobenzoic acid (hereinafter abbreviated as PABA), PABA monoglycerine ester, N,N-dipropoxy PABA ethyl ester, N,N-diethoxy PABA ethyl ester, N,N-dimethyl PABA ethyl ester, N,N-dimethyl PABA butyl ester, N,N-dimethyl PABA ethyl ester, etc); anthranilic acid family ultraviolet light absorber (such as homomenthyl N-acetylanthranilate etc); salicylic acid family ultraviolet light absorber (such as amyl salicylate, menthyl salicylate, homomenthyl salicylate, octyl salicylate, phenyl salicylate, benzyl salicylate, p-isopropanolphenyl salicylate, etc); cinnamic acid family ultraviolet light absorber (such as octyl methoxycinnamate, ethyl 4-isopropylcinnamate, methyl 2,5-diisopropylcinnamate, ethyl 2,4-diisopropylcinnamate, methyl 2,4-diisopropylcinnamate, propyl p-methoxycinnamate, isopropyl p-methoxycinnamate, isoamyl p-methoxycinnamate, octyl p-methoxycinnamate (2-ethylhexyl p-methoxycinnamate), 2-ethoxyethyl p-methoxy cinnamate, cyclohexyl p-methoxycinnamate, ethyl α-cyano-β-phenylcinnamate, 2-ethylhexyl α-cyano-β-phenylcinnamate, glyceryl mono-2-ethylhexanoyl-diparamethoxy cinnamate, etc); benzophenone family ultraviolet light absorber (such as 2,4-dihydroxybenzophenone, 2,2′-dihydroxy-4-methoxybenzophenone, 2,2′-dihydroxy-4,4′-dimethoxybenzophenone, 2,2′,4,4′-tetrahydroxybenzophenone, 2-hydroxy-4-methoxybenzophenone, 2-hydroxy-4-methoxy-4′-methylbenzophenone, 2-hydroxy-4-methoxybenzophenone-5-sulfonate, 4-phenylbenzophenone, 2-ethylhexyl-4′-phenyl-benzophenone-2-carboxylate, 2-hydroxy-4-n-octoxybenzophenone, 4-hydroxy-3-carboxybenzophenone, etc); 3-(4′-methylbenzylidene)-d,l-camphor and 3-benzylidene-d,l-camphor; 2-phenyl-5-methylbenzoxazol; 2,2′-hydroxy-5-methylphenylbenzotriazol, 2-(2′-hydroxy-5′-t-octylphenyl) benzotriazol, 2-(2′-hydroxy-5′-methylphenylbenzotriazol; dibenzalazine; dianisoylmethane; 4-methoxy-4′-t-butyldibenzoylmethane; 5-(3,3-dimethyl-2-norbornylidene)-3-pentane-2-one; dimorpholinopyridazinone; 2-ethylhexyl 2-cyano-3,3-diphenylacrylate; 2,4-bis-{[4-(2-ethylhexyloxy)-2-hydroxy]-phenyl}-6-(4-methoxyphenyl)-(1,3,5)-triazine, and the like.

Examples of the metal ion sequestrant may include 1-hydroxyethane-1, 1-diphosphonic acid, 1-hydroxyethane, 1-diphosphonic acid 4Na salt, disodium edetate, trisodium edetate, tetrasodium edetate, sodium citrate, sodium polyphosphate, sodium metaphosphate, gluconic acid, phosphoric acid, citric acid, ascorbic acid, succinic acid, edetic acid, trisodium hydroxyethyl ethylenediamine triacetate, and the like.

Examples of the amino acid may include neutral amino acid (such as threonine, cysteine, etc); basic amino acid (such as hydroxylysine, etc) and the like. Examples of the amino acid derivative may include sodium acyl sarcosinate (sodium lauroyl sarcosinate), acyl glutamate, sodium acyl β-alanine, glutathione, pyrrolidone carboxylate, and the like.

Examples of the organic amine may include monoethanolamine, diethanolamine, triethanolamine, morpholine, triisopropanolamine, 2-amino-2-methyl-1,3-propanediol, 2-amino-2-methyl-1-propanol, and the like.

Examples of the polymer emulsion may include acrylic resin emulsion, ethyl polyacrylate emulsion, solution of acrylic resin, polyacrylalkylester emulsion, polyvinyl acetate resin emulsion, natural rubber latex, and the like.

Examples of the pH modifier may include buffer such as lactic acid-sodium lactate, citric acid-sodium citrate, succinic acid-sodium succinate, and the like.

Examples of the vitamins may include vitamin A, B1, B2, B6, C, E and derivatives thereof, pantothenic acid and derivatives thereof, biotin, and the like.

Examples of the anti-oxidant may include tocopherols, dibutyl hydroxy toluene, butyl hydroxy anisole, and gallic acid esters, and the like.

Examples of the anti-oxidant aid may include phosphoric acid, citric acid, ascorbic acid, maleic acid, malonic acid, succinic acid, fumaric acid, cephalin, hexamethaphosphate, phytic acid, ethylenediaminetetraacetic acid, and the like.

Examples of other containable compositions may include an antiseptic agent (such as ethylparaben, butylparaben, chlorphenesin, 2-phenoxyethanol, etc); antiphlogistic (such as glycyrrhizinic acid derivatives, glycyrrhetic acid derivatives, salicylic acid derivatives, hinokitiol, zinc oxide, allantoin, etc); a skin-whitening agent (such as placental extract, saxifrage extract, arbutin, etc); various extracts (such as phellodendron bark (cork tree bark), coptis rhizome, lithospermum, peony, swertia herb, birch, sage, loquat, carrot, aloe, mallow, iris, grape, coix seed, sponge gourd, lily, saffron, cnidium rhizome, ginger, hypericum, restharrow, garlic, red pepper, citrus unshiu, Japanese angelica, seaweed, etc); an activator (such as royal jelly, photosenstizer, cholesterol derivatives, etc); a blood circulation promotion agent (such as nonylic acid vanillylamide, nicotine acid benzyl ester, nicotine acid β-butoxyethyl ester, capsaicin, zingerone, cantharides tincture, ichthammol, tannic acid, α-borneol, tocopheryl nicotinate, meso-inositol hexanicotinate, cyclandelate, cinnarizine, tolazoline, acetylcholine, verapamil, cepharanthine, γ-oryzanol, etc); an antiseborrheric agent, (such as sulfur, thianthl, etc); an anti-inflammatory agent (such as tranexamic acid, thiotaurine, hypotaurine, etc), and the like.

The composition of the present disclosure further may include, as necessary, caffeine, tannin, verapamil, tranexamic acid and derivatives thereof; various crude drug extracts such as licorice, Chinese quince, Pyrola japonica and the like; drugs such as tocopherol acetate, glycyrrhetinic acid, glycyrrhizic acid and derivatives thereof, or salts thereof; skin-whitening agents such as vitamin C, magnesium ascorbyl phosphate, ascorbic acid glucoside, arbutin, kojic acid and the like; amino acids such as arginine and lysine and the like and derivatives thereof.

Phase Constitution:

The cleansing composition according to the first embodiment may have a desired phase constitution depending on the purpose and/or application thereof. The cleansing composition may take various forms, such as an aqueous composition, an oily component, an oil-in-water type composition, a water-in-oil type composition, a bicontinuous composition, an oil-water separation-type composition, a fatty acid soap-containing composition, or the like.

An oil-water separation-type composition has a two-layer constitution wherein the oil phase and the aqueous phase are separated into upper and lower layers in a stationary state. Since an oil-water separation-type composition can contain large amounts of oily components, cleansability can be improved. Also, the presence of the aqueous phase can improve the feel upon use. Further, the content of surfactants can be reduced, and thus, the composition can be employed for leave-on (wipe-off) type applications. In cases where the composition is an oil-water two-layer separation type, the mass ratio between the aqueous phase and the oil phase is preferably 0.25 parts or greater, more preferably 0.5 parts or greater, further preferably 1 part or greater, of the aqueous phase with respect to 1 part of the oil phase. If the mass ratio of the aqueous phase is less than 0.25, the refreshed feeling upon use may be impaired. The mass ratio between the aqueous phase and the oil phase is preferably 4 parts or less, more preferably 2.5 parts or less, further preferably 2 parts or less, of the aqueous phase with respect to 1 part of the oil phase. If the mass ratio of the aqueous phase exceeds 4, component (A) may precipitate out and transparency may deteriorate.

PH:

In cases where the cleansing composition according to the first embodiment includes an aqueous phase, it is preferred that the pH of the aqueous phase is preferably 5 or higher, more preferably 5.5 or higher. If the pH is below 5, component (A) may likely precipitate out and transparency may deteriorate. The pH of the aqueous phase is preferably 8 or lower, more preferably 7.5 or lower. If the pH exceeds 8, irritation to the skin may become too strong.

Appearance:

The cleansing composition according to the first embodiment may have high transparency. Thus, the cleansing composition can be placed in a transparent container. By being able to use a transparent container, in cases, for example, where the cleansing composition is an oil-water two-layer separation-type, the emulsification state created by shaking can be visually observed.

In cases where the cleansing composition is an oil-water two-layer separation-type, it is possible to obtain a composition wherein both the aqueous phase and the oil phase are non-turbid, and the interface between the aqueous phase and the oil phase is clear.

Cleansability:

The cleansability of the cleansing composition of the present disclosure will be described below. The cleansing composition according to the first embodiment can be suitably used, for example, for cosmetic removal cleansing agents, hand soaps, body soaps, hair cleansing agents, kitchen cleansing agents, and the like.

The cleansing composition of the present disclosure can be used as a cleansing agent for removing dyes (e.g., acidic dyes) staining the skin. For example, the cleansing composition of the present disclosure can be used as a cleanser applicable to tinted cosmetics. Typically, acidic dyes (anion dyes) are used for dyes in tinted cosmetics. Acidic dyes are thought to form bonds with proteins in the skin by chemical interaction (e.g., ionic interaction). By including component (A), the cleansing composition of the present disclosure has excellent cleansability against such acidic dyes.

The cleansing composition of the present disclosure can be used as a cleansing agent that is rinsed off with water, or can be used as a cleansing agent that does not require rinsing with water (i.e., a leave-on-type composition). In cases where the composition is a leave-on type composition, the cleansing composition can be, for example, applied or dropped onto an object being cleansed (e.g., the skin) and rubbed, and then cleansing can be finished simply by wiping off the composition with a fiber product such as tissue paper or a pad. Alternatively, a fiber product may be impregnated with the cleansing composition, and an object being cleansed (e.g., the skin) may be rubbed with the fiber product to remove components that need to be removed. Particularly, even in cases where a dye is to be removed from the skin (e.g., a tinted cosmetic is to be removed) as described above, the present cleansing composition can be used as a leave-on-type composition. For example, disodium cocoamphodiacetate has cleansability against tinted cosmetics but is highly irritant to the skin, and thus needs to be rinsed off with water when removing tinted cosmetics. In contrast, the cleansing composition of the present disclosure can be used as a leave-on-type cleanser applicable to tinted cosmetics.

Method of Use:

In cases where the cleansing composition according to the first embodiment is an oil-water two-layer separation-type composition, a container holding the cleansing composition may be shaken before extracting the composition to bring the composition into a temporarily emulsified state (including a quasi-emulsified state), and then the cleansing composition may be taken out from the container.

The number of times to shake the container to bring the composition into an emulsified state may be, for example, preferably 20 times or fewer, more preferably 15 times or fewer, further preferably 10 times or fewer. It is preferred that the temporarily emulsified state created by the shaking operation lasts for a predetermined time. For example, it is preferred that the temporarily emulsified state lasts for preferably 10 seconds or longer, more preferably 15 seconds or longer, further preferably 20 seconds or longer. Also, it is preferred that, when the composition returns from the temporarily emulsified state to the oil-water two-layer separated state by being left standing, the cleansing composition is not turbid but is transparent. It is also preferred that the interface between the oil phase and the aqueous phase is clear.

Manufacturing Method:

A method for manufacturing the cleansing composition of the present disclosure will be described below. The method for manufacturing the cleansing composition of the present disclosure is not particularly limited, and the composition can be prepared by generally known methods. For example, the cleansing composition can be prepared by mixing the aforementioned components. In cases where component (A) is oil-soluble, component (A) may be dissolved in component (B) to manufacture the cleansing composition. In cases where component (A) is water-soluble, component (A) may be dissolved in component (D) and/or component (E) to manufacture the cleansing composition.

There may be cases where it is difficult, or utterly impractical, to directly define the phase constitution etc. of the cleansing composition of the present disclosure based on the compositional makeup thereof. In such circumstances, it should be permissible to define the cleansing composition of the present disclosure according to methods for producing the same.

### EXAMPLES

The cleansing composition of the present disclosure will be described below by way of examples. The cleansing composition of the present disclosure is, however, not limited to the following examples. The following describes examples wherein the cleansing compositions according to the respective Test Examples are used for cleansing off cosmetics, but the composition of the present disclosure is not limited to use for cosmetics. The unit employed for indicating the content by percentage of each component shown in the Tables is percent by mass (mass %).

### Test Examples 1 to 8

Oil-water two-layer separation-type cleansing compositions were prepared, and their cleansability against tinted cosmetic was tested. Tables 1 and 2 show the compositional makeup and results for each Test Example.

In each of the Test Examples, the oil phase and the aqueous phase were prepared separately and then the two phases were mixed, to prepare an oil-water two-layer-type cleansing composition. In the tables below, the “oil phase” and the “aqueous phase” indicate the respective phases to which the various components were blended at the time of preparation of the cleansing composition. It should thus be added that, by shaking and/or during standing (in a stationary state), components blended to the oil phase may migrate to the aqueous phase, and/or components blended to the aqueous phase may migrate to the oil phase.

Cleansability Against Tinted Cosmetic:

The L*a*b* color space of an inner arm section for applying a tinted cosmetic was measured with a colorimeter (Spectrophotometer SE7700 from Nippon Denshoku Industries Co., Ltd.). The a* value found here is defined as “a (Base)”. Next, a tinted cosmetic was applied to the inner arm section where the color space was measured, and the cosmetic was left for 20 minutes to stain the skin. Excess tinted cosmetic was wiped off. For the tinted cosmetic, a tinted cosmetic containing tetrafluoro-tetrabromofluorescein as an acidic cosmetic was used. The L*a*b* color space of the section stained with the tinted cosmetic was measured. The a* value found here is defined as “a (Blank)”. The oil-water two-layer separation-type cleansing composition according to each Test Example was emulsified, and a cotton pad impregnated with 1 g of the emulsified cleansing composition was used to rub the respective sections stained with the tinted cosmetic 20 times with uniform force. After wiping off the cleansing composition, the L*a*b* color space of the stained section after cleansing was measured. The a* value found here is defined as “a (Sample)”. The cleansability of the cleansing composition according to each Test Example was evaluated according to “cleansing rate (%)” calculated from the following equation. The formula for calculating the cleansing rate and the evaluation criteria are shown below.

$$Cleansing rate ( \% ) = \frac{1 - ( a ( Sample ) - a ( Base ) )}{a ( Blank ) - a ( Base )} \times 100 \qquad [ Math . 1 ]$$

A: Cleansing rate was 75% or higher.
B: Cleansing rate was 60% or higher to below 75%.
D: Cleansing rate was 45% or higher to below 60%.
D: Cleansing rate was below 45%.

The cleansing composition according to Test Example 1, which did not contain component (A), had poor cleansability against tinted cosmetic. In contrast, Test Examples 2 to 4, which contained lauryl dimethylamine oxide as component (A), were capable of improving cleansability against tinted cosmetic. Also, Test Examples 5 to 7, which contained stearyl dimethylamine oxide as component (A), were capable of improving cleansability against tinted cosmetic. These results show that adding component (A) can improve cleansing effects against dyes adhering to the skin.

Test Example 8, which contained N,N,N′,N′-tetrakis(2-hydroxypropyl)ethylenediamine instead of component (A), exhibited an improvement in cleansability against tinted cosmetic. However, the evaluation rating of Test Example 8 was equivalent to Test Example 2, whose content by percentage of component (A) was less than one-third that of the ethylenediamine compound. When compared with Test Examples 4 and 7, whose content by percentage of component (A) was equivalent to that of the ethylenediamine compound in Test Example 8, Test Examples 4 and 7 had considerably higher cleansability. This shows that component (A) can achieve higher cleansability against tinted cosmetic with a smaller amount of addition, compared to the ethylenediamine compound of Test Example 8.

In Test Examples 2 to 7, it was found that increasing the blending amount of component (A) tended to improve cleansability. This suggests that the blending amount of component (A) with respect to the mass of the composition is preferably 0.2% by mass or greater, more preferably 0.4% by mass or greater, further preferably 0.6% by mass or greater, further preferably 0.8% by mass or greater, further preferably 1% by mass or greater, further preferably 1.2% by mass or greater, further preferably 1.5% by mass or greater.

The cleansing compositions according to Test Examples 2 to 7 were capable of being used as leave-on-type compositions.

TABLE 1

| Test Example | | 1 | 2 | 3 | 4 |
|---|---|---|---|---|---|
| Oil | (B) Dimethicone | 24 | 24 | 24 | 24 |
| phase | (B) Isododecane | 8 | 8 | 8 | 8 |
| Aqueous | (E) Ethanol | 3 | 3 | 3 | 8 |
| phase | (E) 1,3-Butylene glycol | 8 | 8 | 8 | 8 |
| | (A) Lauryl dimethylamine oxide | — | 0.3 | 0.6 | 0.9 |
| | (A) Stearyl dimethylamine oxide | — | — | — | — |
| | N,N,N′,N′-tetrakis(2- | — | — | — | — |
| | hydroxypropyl)ethylenediamine | | | | |
| | (C3) Lauryldimethylaminoacetic | 0.05 | 0.05 | 0.05 | 0.05 |
| | acid betaine | | | | |
| | (G) Sodium chloride | 0.5 | 0.5 | 0.5 | 0.5 |
| | (H) Phenoxyethanol | 0.2 | 0.2 | 0.2 | 0.2 |
| | (H) Citric acid | 0.01 | 0.01 | 0.01 | 0.01 |
| | (H) Sodium citrate | 0.09 | 0.09 | 0.09 | 0.09 |
| | (H) EDTA-2Na•2H₂O | 0.02 | 0.02 | 0.02 | 0.02 |
| | (D) Ion-exchanged water | Balance | Balance | Balance | Balance |
| Total | | 100 | 100 | 100 | 100 |
| Evaluation | Cleansability against tinted cosmetic | D | C | B | A |

TABLE 2

| Test Example | | 5 | 6 | 7 | 8 |
|---|---|---|---|---|---|
| Oil | (B) Dimethicone | 24 | 24 | 24 | 24 |
| phase | (B) Isododecane | 8 | 8 | 8 | 8 |
| Aqueous | (E) Ethanol | 8 | 3 | 3 | 3 |
| phase | (E) 1,3-Butylene glycol | 8 | 8 | 8 | 8 |
| | (A) Lauryl dimethylamine oxide | — | — | — | — |
| | (A) Stearyl dimethylamine oxide | 0.3 | 0.6 | 0.9 | — |
| | N,N,N′,N′-tetrakis(2- | — | | | 1 |
| | hydroxypropyl)ethylenediamine | | | | |
| | (C3) Lauryldimethylaminoacetic | 0.05 | 0.05 | 0.05 | 0.05 |
| | acid betaine | | | | |
| | (G) Sodium chloride | 0.5 | 0.5 | 0.5 | 0.5 |
| | (H) Phenoxyethanol | 0.2 | 0.2 | 0.2 | 0.2 |
| | (H) Citric acid | 0.01 | 0.01 | 0.01 | 0.01 |
| | (H) Sodium citrate | 0.09 | 0.09 | 0.09 | 0.09 |
| | (H) EDTA-2Na•2H₂O | 0.02 | 0.02 | 0.02 | 0.02 |
| | (D) Ion-exchanged water | Balance | Balance | Balance | Balance |
| Total | | 100 | 100 | 100 | 100 |
| Evaluation | Cleansability against tinted cosmetic | B | B | A | C |

The cleansing composition of the present invention have been described according to the foregoing embodiments and examples, but the invention is not limited to the foregoing embodiments and examples and may encompass various transformations, modifications, and improvements made to the various disclosed elements (including elements disclosed in the Claims, Description, and Drawings) within the scope of the invention and according to the fundamental technical idea of the present invention. Further, various combinations, substitutions, and selections of the various disclosed elements are possible within the scope of the claims of the invention.

Further issues, objectives, and embodiments (including modifications) of the present invention are revealed also from the entire disclosure of the invention including the Claims.

The numerical ranges disclosed herein are to be construed in such a manner that arbitrary numerical values and ranges falling within the disclosed ranges are treated as being concretely described herein, even where not specifically stated.

### INDUSTRIAL APPLICABILITY

Some or all of the foregoing embodiments may be described as in the following additional items, although not limited thereto. The various additional items may be employed in combination with the claim(s) in the Scope of Claims.

{Additional Item 1}

A cleansing method for removing a dye from the skin by using a cleansing composition containing a trialkylamine oxide.

{Additional Item 2}

A cleansing method using a cleansing composition containing a trialkylamine oxide represented by Chem. 1.

{Additional Item 3}

A cleansing method using the cleansing composition as a leave-on-type composition.

### INDUSTRIAL APPLICABILITY

The cleansing composition of the present disclosure can be suitably used for cleansing the skin. Particularly, the composition of the present disclosure can be suitably used for cleansing to remove cosmetics on the skin.

1. A dye cleansing composition for removing a dye from the skin, the cleansing composition comprising a trialkylamine oxide represented by Chem. 1: 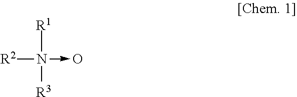
(in the chemical formula represented by Chem. 1, two of R¹, R², and R³ are each a C₁₋₄ alkyl group, and one is a C₁₂₋₂₁ alkyl group).

2. The cleansing composition according to claim 1, wherein the dye is an acidic dye.

3. The cleansing composition according to claim 1, wherein a content by percentage of the trialkylamine oxide is from 0.2 to 4% by mass with respect to the mass of the composition.

4. The cleansing composition according to claim 1, wherein:
the composition comprises an oil phase and an aqueous phase; and
in a stationary state, the oil phase and the aqueous phase are primarily not emulsified.

5. The cleansing composition according to claim 4, wherein:
a content by percentage of the oil phase is from 20 to 80% by mass with respect to the mass of the composition; and
a content by percentage of the aqueous phase is from 20 to 80% by mass with respect to the mass of the composition.

6. The cleansing composition according to claim 4, wherein the aqueous phase has a pH of from 4 to 8.

7. The cleansing composition according to claim 1, wherein a content by percentage of a surfactant is 0.2% by mass or less with respect to the mass of the composition.

8. The cleansing composition according to claim 1, further comprising an alkylbetaine-type surfactant.

9. The cleansing composition according to claim 1, further comprising from 1 to 20% by mass of a water-soluble alcohol with respect to the mass of the composition.

10. The cleansing composition according to claim 1, further comprising from 15 to 50% by mass of a volatile oily component with respect to the mass of the composition.

11. The cleansing composition according to claim 10, wherein the volatile oily component is at least one selected from the group consisting of hydrocarbon oils, ester oils, and silicone oils.

12. The cleansing composition according to claim 1, further comprising from 0.1 to 5% by mass of a salt with respect to the mass of the composition.

13. The cleansing composition according to claim 1, further comprising from 0.05 to 5% by mass of a polyol derivative with respect to the mass of the composition, wherein
the polyol derivative is a glycerin derivative represented by Chem. 2 and/or a glycol derivative represented by Chem. 3: 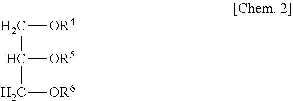
(in the chemical formula represented by Chem. 2, one of R⁴, R⁵, and R⁶ is a C₄₋₁₅ alkyl group, alkenyl group or acyl group, and two are each a hydrogen atom); 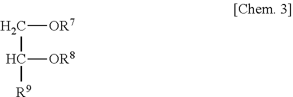
(in the chemical formula represented by Chem. 3, one of R⁷ and R⁸ is a C₁₀₋₂₀ alkyl group, alkenyl group or acyl group, and the other is a hydrogen atom; and R⁹ is a C₁₋₄ alkyl group, alkenyl group or acyl group, or a hydrogen atom).

14. The cleansing composition according to claim 13, wherein the glycerin derivative includes ethylhexylglycerin and/or hexylglycerin.

15. The cleansing composition according to claim 13, wherein the glycol derivative includes at least one selected from the group consisting of propylene glycol laurate, propylene glycol stearate, and propylene glycol isostearate.

16. The cleansing composition according to claim 1, wherein the cleansing composition is a cleansing composition for cosmetics.

17. The cleansing composition according to claim 1, wherein the cleansing composition is a leave-on-type composition.

18. The cleansing composition according to claim 4, wherein a content by percentage of a surfactant is 0.2% by mass or less with respect to the mass of the composition.

19. The cleansing composition according to claim 18, further comprising an alkylbetaine-type surfactant.

20. The cleansing composition according to claim 19, further comprising from 0.05 to 5% by mass of a polyol derivative with respect to the mass of the composition, wherein
the polyol derivative is a glycerin derivative represented by Chem. 4 and/or a glycol derivative represented by Chem. 5: 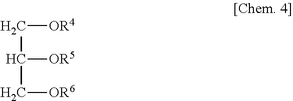
(in the chemical formula represented by Chem. 4, one of R⁴, R⁵, and R⁶ is a C₄₋₁₅ alkyl group, alkenyl group or acyl group, and two are each a hydrogen atom); 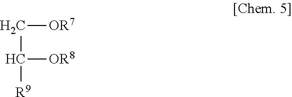
(in the chemical formula represented by Chem. 5, one of R⁷ and R⁸ is a C₁₀₋₂₀ alkyl group, alkenyl group or acyl group, and the other is a hydrogen atom; and R⁹ is a C₁₋₄ alkyl group, alkenyl group or acyl group, or a hydrogen atom).
